(19) United States
(12) Patent Application Publication
(10) Pub. No.: US 2021/0330915 A1
(43) Pub. Date: Oct. 28, 2021

(54) MOUTHPIECE CONNECTOR AND BREATHING SYSTEM

(71) Applicant: John Neil Haugsrud, Arlington, VT (US)

(72) Inventor: John Neil Haugsrud, Arlington, VT (US)

(21) Appl. No.: 17/240,170

(22) Filed: Apr. 26, 2021

Related U.S. Application Data
(60) Provisional application No. 63/016,627, filed on Apr. 28, 2020.

(51) Int. Cl. A61M 16/04; A61M 16/20; A61M 16/06

(52) U.S. Cl. CPC A61M 16/049; A61M 16/0463; A61M 16/06; A61M 16/205; A61M 16/204

(57) ABSTRACT

A mouthpiece, the mouthpiece is made of a conformable material and has a breathing cavity extending there through. The mouthpiece has a pair of teeth slots for holding the mouthpiece between teeth and an ergonomic shape that seals with lips. The mouthpiece is a connector that allows for easy attachment of a breathing tube. The mouthpiece can be combined with various tubes, valves and filters to create a variety of breathing systems.

### CROSS-REFERENCE TO RELATED APPLICATIONS

This application claims the benefit of priority of U.S. Provisional Patent Application No. 63/016,627, filed Mar. 28, 2020, the disclosure of which is herein incorporated by reference. This patent application is related to U.S. Design patent application Ser. No. 29/732,891, filed on Mar. 28, 2020, and titled “Mouthpiece”, which is incorporated herein by reference.

### FIELD

The present invention generally relates to sanitary breathing. More specifically, the invention relates to a mouthpiece connector used as part of a sanitary breathing system.

### BACKGROUND

Airborne contaminants are a problem in many situations such as the generation of fine particles in building trades, toxic particle generation in certain manufacturing settings and biological hazards in hospitals. The most common item used to protect the lungs from contaminants is a mask. Masks can protect both the wearer's lungs from external contaminants and other individuals from contaminants that may be emanating from the wearer's lungs. Masks have a number of problems leading to less than complete protection for all. One issue is that masks only partially seal with the nose, mouth and face and therefore always allow for some contaminants to either escape from the person or vice versa enter the breathing passages of the person wearing the mask. This problem is exacerbated by facial hair such as a thick beard. Other deficiencies are that masks get moist with extended use, as well as get dirty from touching. For biological contaminants, probably the biggest problem with masks is that their use still spreads contamination from the wearer's exhale because the mask does not seal well with the face.

The present invention aims to eliminate the above noted deficiencies of masks and provide for a more sanitary breathing system.

### SUMMARY

In one implementation, the present disclosure is directed to a device for redirecting air flow from a human's mouth. The mouthpiece is formed of a conformable and pliable material that has a mouth end, exterior end and opposed sealing surfaces. The mouthpiece further includes a breathing cavity extending through the mouthpiece from the mouth end to the exterior end. The breathing cavity is adaptable at one end to accept a variety of different breathing attachments. The opposed sealing surfaces are spaced-apart furthest apart from the center along the breathing cavity and tapering to meet at their edges. A pair of teeth slots are proximate the mouth end, with each teeth slot extending from the sealing surfaces and into the breathing cavity.

In another implementation, the present disclosure is directed to a breathing system. The breathing system comprises a mouthpiece, a breathing tube and one or more valves and filters to help purify and redirect the air.

### BRIEF DESCRIPTION OF DRAWINGS

For the purposes of illustrating the invention, the drawings show aspects of one or more embodiments of the invention. However, it should be understood that the present invention is not limited to the precise arrangements and instrumentalities shown in the drawings, wherein:

FIG. 1 is a top, front perspective view of one exemplary embodiment of a mouthpiece according to the present invention;

FIG. 2 is a bottom, front perspective view of the mouthpiece in FIG. 1;

FIG. 3 is a front and back elevational view of the mouthpiece in FIG. 1;

FIG. 4 is a right-side and left-side elevational view of the mouthpiece in FIG. 1;

FIG. 5 is a top view of the mouthpiece in FIG. 1;

FIG. 6 is a bottom view of the mouthpiece in FIG. 1;

FIG. 7 is a sectional view along line 7-7 of FIG. 2 illustrating taper of the breathing cavity of the mouthpiece in FIG. 1;

FIG. 8 is the sectional view of FIG. 7 with a breathing tube now inserted into the breathing cavity;

FIG. 9 is a schematic diagram illustrating placement of the mouthpiece of FIG. 1 in an open mouth;

FIG. 10 is a schematic diagram illustrating sealing of lips around the mouthpiece of FIG. 9 when a mouth is closed;

FIG. 11 is a sectional view along line 11-11 of FIG. 10 showing placement of the mouthpiece during use in a mouth;

FIG. 12 is a schematic diagram showing the mouthpiece of FIG. 1 now integrated with a breathing tube and filter to create one embodiment of a sanitary breathing system;

FIG. 13 is a side view of the mouthpiece of FIG. 1 now integrated with various other components to create a another embodiment of a sanitary breathing system; and

FIG. 14 is a schematic diagram showing the sanitary breathing system of FIG. 13 in use by a person.

### DETAILED DESCRIPTION

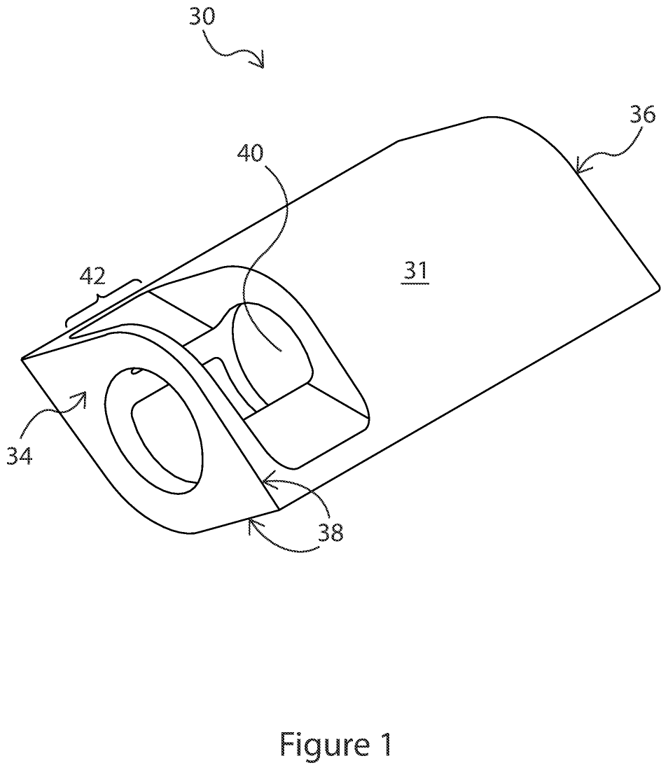

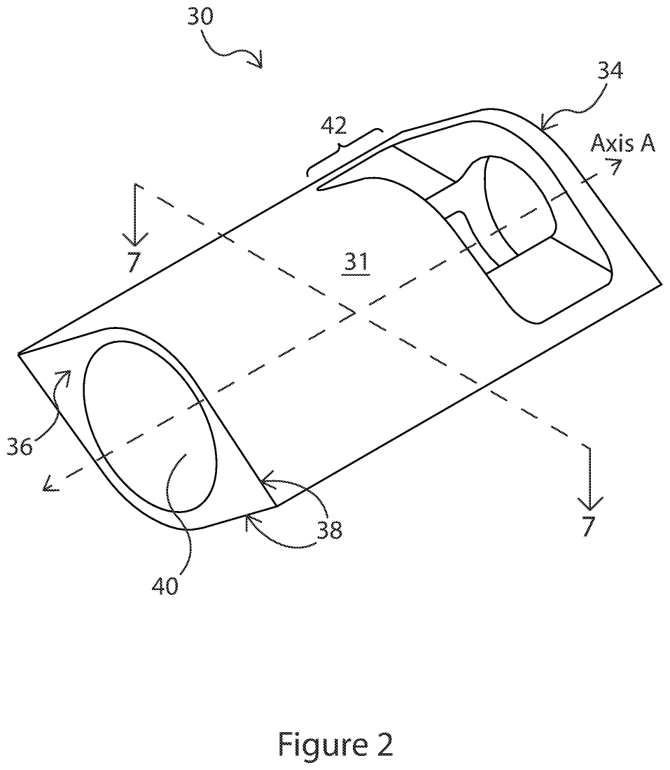

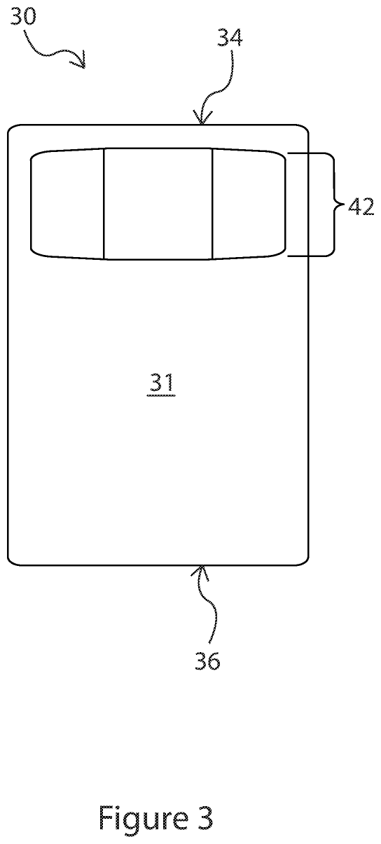

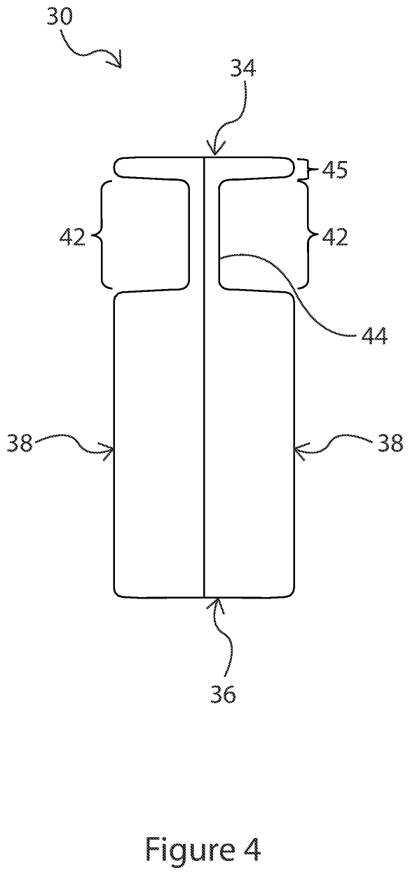

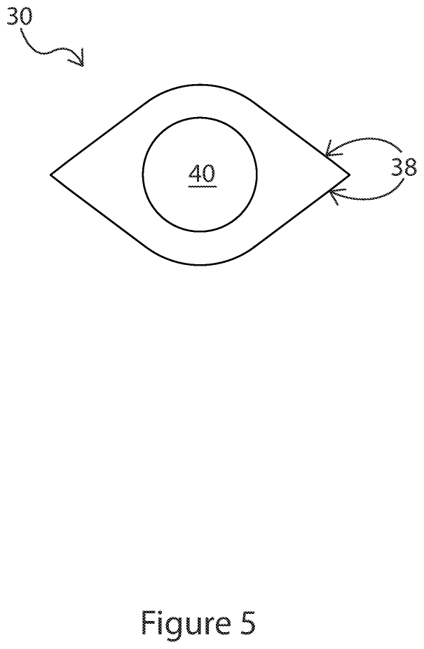

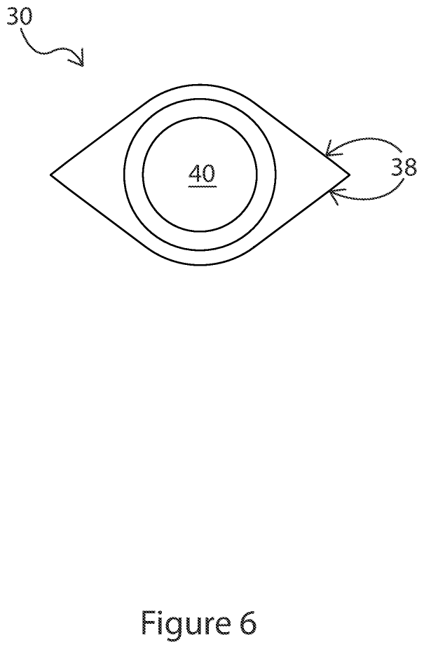

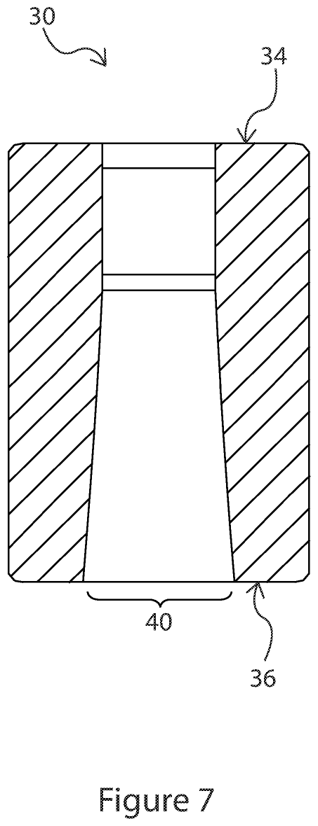

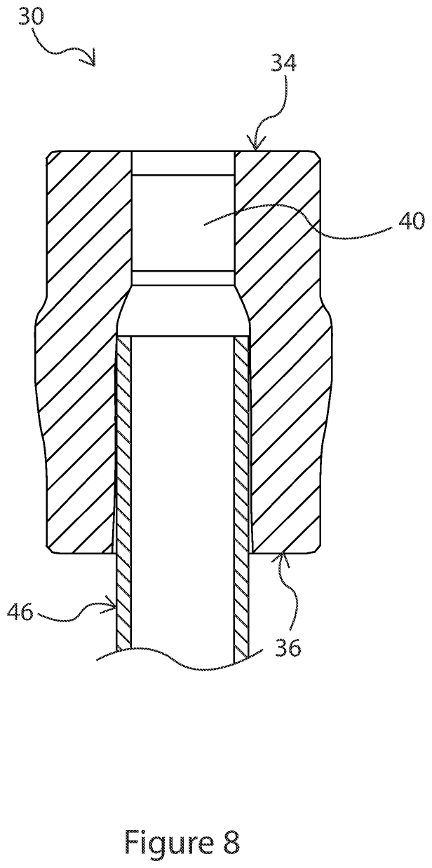

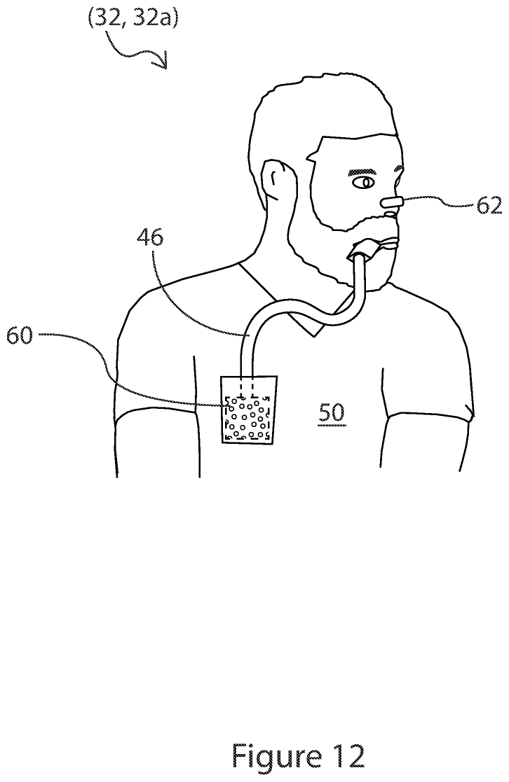

Mouthpiece 30 is illustrated in FIGS. 1-11. Mouthpiece 30 may be integrated with various other components to create a sanitary breathing system 32 as shown in FIGS. 12-14. Mouthpiece 30 comprises a body portion 31 having mouth end 34, exterior end 36 and opposing sealing surfaces 38, FIGS. 1-6. Opposed sealing surfaces 38 are two convex sealing surfaces that meet at an acute angle. Sealing surfaces 38 have a generally bi-convex lenticular cross-section perpendicular to the breathing cavity. Opposed sealing surfaces 38 form a cone cross-section where they meet. Mouthpiece 30 further comprises a breathing cavity 40 (a.k.a. breathing passage) extending through the mouthpiece from mouth end 34 to exterior end 36. Breathing cavity 40 is preferably along central axis A of mouthpiece 30. Sealing surfaces 38 are spaced furthest apart from central axis A along breathing cavity 40. A pair of teeth slots 42 are positioned proximate mouth end 34. Teeth slots 42 are sized to accept human molar teeth 54. Teeth slots 42 are perpendicular to central axis A, one slot extending from each sealing surface 38 into breathing cavity 40. Teeth slots 42 run parallel to each other with a tooth separation barrier 44 separating the teeth slots. By having the teeth slots 42 distanced a small distance away from mouth end 34; a tooth anchoring strip 45 is created. Tooth anchoring strip 45 holds mouthpiece 30 within mouth 52 even if teeth are not completely closed. Breathing cavity 40 may have a taper that narrows the breathing cavity diameter from exterior end 36 to teeth slots 42. This taper allows for the acceptance of breathing tubes 46 of various diameters into mouthpiece 30. The sections in FIG. 7 and FIG. 8 show how when a breathing tube 46 may be pushed into breathing cavity 40 that the breathing cavity expands near the end of the breathing tube to snuggly hold the breathing tube. At exterior end 36, breathing cavity 40 may instead have other components such as an adhesive, screw attachment and tube fittings to secure a breathing tube 46 to mouthpiece 30. For example, the tube fitting may be a Leur fitting.

It is critical that mouthpiece 30 be fabricated from a conformable material in the range of 30 to 90 Shore A hardness. By having mouthpiece 30 in this hardness range, the mouth piece is first and foremost comfortable for the user. A hard mouthpiece would be unpleasant to keep in the mouth for long periods of time. Second, this hardness allows the users teeth to grip mouthpiece 30 tightly without breaking the mouthpiece or conversely breaking the teeth. Third, this hardness allows the mouthpiece to conform to different size breathing tubes or fittings inserted into the mouthpiece and hold them snuggly so they do not fall out.

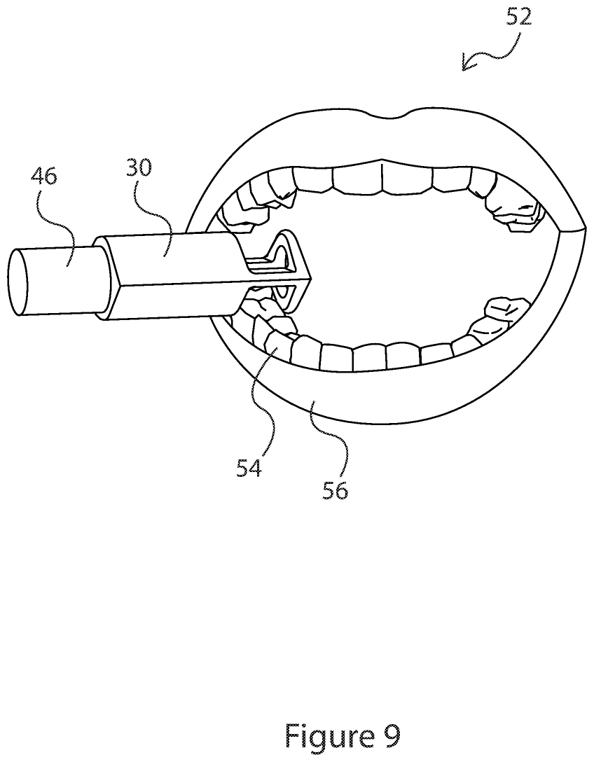

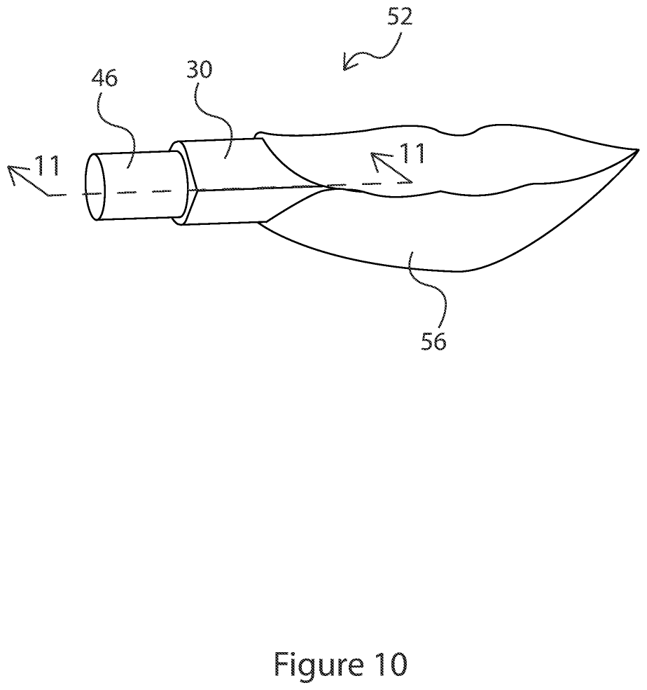

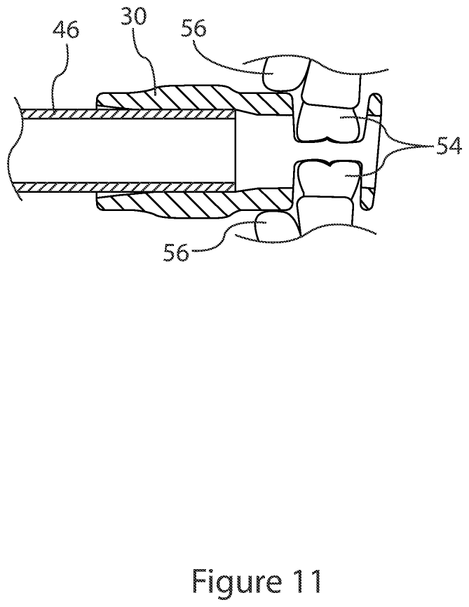

Use of mouthpiece 30 is shown in FIG. 9. User 50 opens mouth 52 and aligns teeth slots 42 with molar teeth 54 in the side of the mouth. User 50 then bites down on tooth separation barrier 44. Lips 56 are closed to seal with sealing surface 38, FIG. 10. FIG. 11 shows how mouthpiece 30 is positioned relative to teeth 54 and lips 56. User 50 may also have mouthpiece 30 reside in mouth 52 with lips 56 closed, but because teeth 54 are partially within teeth slots 42 the teeth do not need to be pressing down on the mouthpiece and the mouthpiece will still be held securely within the mouth by tooth anchor strip 45.

Mouthpiece 30 is used as part of a breathing system 32 (32a, 32b). In one embodiment, FIG. 12, breathing system 32a comprises mouth piece 30, breathing tube 46 inserted into the mouthpiece and filter 60 on the distal end of the breathing tube. A nose clip 62 or nose plugs are used to seal the nasal passages, allowing air flow to only enter and exit through the user's mouth 50 via the mouthpiece system. When filter 60 is on breathing tube 46, only clean air enters the user.

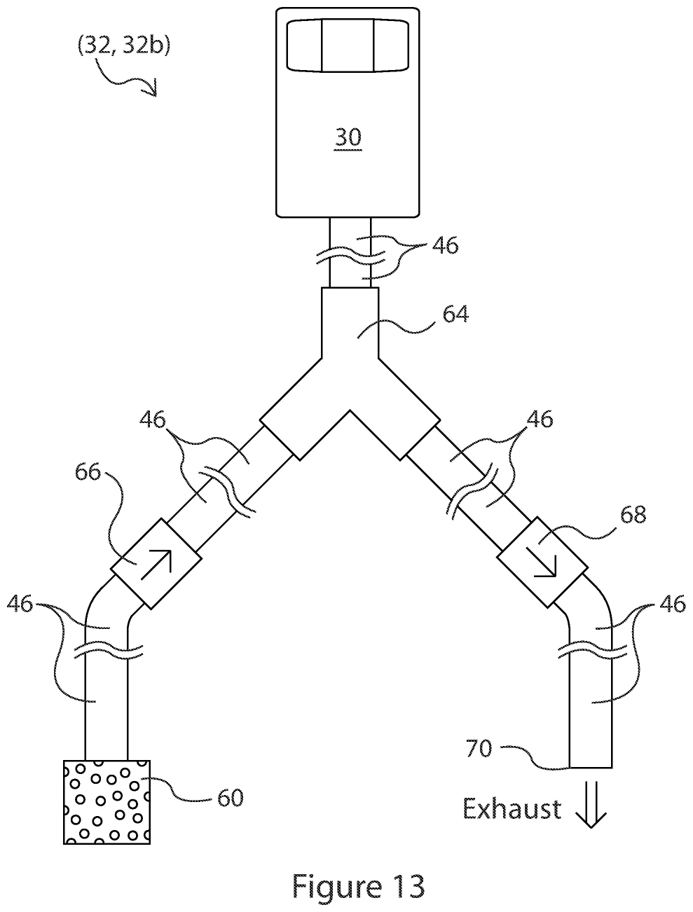

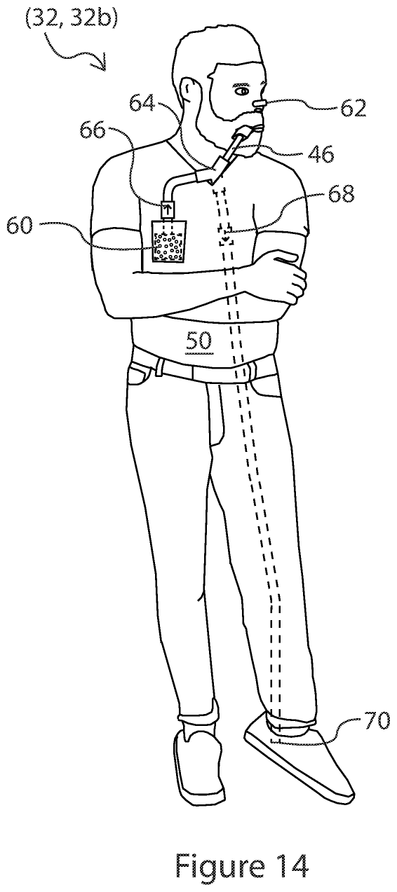

In a second embodiment, FIG. 13, mouthpiece 30 is combined with additional elements to form another breathing system 32b. Here mouthpiece 30 has a breathing tube 46 inserted into breathing cavity 40. A splitting-connector 64 splits the air pathway. On one side of the split air path is integrated a one-way air intake valve 66 to allow air to flow into the user's mouth. On the other side of the split air path is integrated a one-way air exhaust valve 68 to allow air to flow out of the user's mouth. At the end of the air pathway prior to intake valve 66 is an air filter 60 that purifies the air and excludes all contaminates from entering the user's breathing system. At the exhaust end 70, after the air exhaust valve, the air may be exhausted with or without another filter depending on the application. Breathing system 32b is shown integrated with a user in FIG. 14 in one exemplary configuration.

The main purpose of this connector mouthpiece 30 is to allow the wearer the flexibility to create a system that will help them to receive dry (filtered) air and with the use of a spitting-connector to another breathing tube 46 to direct the exhale to a desired location (filter, floor, outside). Such a system can protect both the wearer and surrounding people. One desirable feature of mouthpiece 30 and system 32 is that the mouthpiece can provide much better sealing than a mask for individuals with facial hair. By exhausting air at a place away from the face, one is less likely to fog goggles that may be worn for further protection. Eating and drinking can be easier and more sanitary. Mouthpiece 30 can be more easily attached and removed than a mask and one does not need to touch the mouthpiece directly. The mouthpiece can be placed in and removed from the mouth by holding only the breathing tube. The mouthpiece can also be connected to positive airflow to aid with breathing.

While several embodiments of the invention, together with modifications thereof, have been described in detail herein and illustrated in the accompanying drawings, it will be evident that various further modifications are possible without departing from the scope of the invention. The scope of the claims should not be limited by the preferred embodiments set forth in the examples, but should be given the broadest interpretation consistent with the description as a whole.

What is claimed is:

1) A device for redirecting air flow from a human having a mouth, the mouth having an oral cavity, lips and teeth; comprising:
a) a mouthpiece having a body portion with a mouth end, exterior end and opposed sealing surfaces;
b) a breathing cavity extending through the mouthpiece from the mouth end to the exterior end;
c) the opposed sealing surfaces spaced-apart furthest along the breathing cavity and tapering to meet at their edges; and
d) a pair of teeth slots proximate the mouth end, each teeth slot extending from the sealing surfaces and into the breathing cavity.

2) The device as recited in claim 1, wherein the mouth piece is formed of a conformable material.

3) The device as recited in claim 2, wherein the conformable material is in the range of Shore A of 30 to 90.

4) The device as recited in claim 1, wherein the sealing surfaces are two convex sealing surfaces.

5) The device as recited in claim 1, wherein the convex sealing surfaces meet in an acute angle.

6) The device as recited in claim 1, wherein the sealing surfaces form at cone cross-section where they meet.

7) The device as recited in claim 1, wherein the sealing surfaces have a bi-convex lenticular cross-section perpendicular to the breathing cavity.

8) The device as recited in claim 1, wherein the teeth slots are sized to accept human teeth.

9) The device as recited in claim 1, wherein the teeth slots are parallel to each other.

10) The device as recited in claim 1, wherein the teeth slots are perpendicular to the breathing cavity.

11) The device as recited in claim 1, further comprising a tooth separation barrier between the two teeth slots for keeping upper and lower teeth separated.

12) The device as recited in claim 1, further comprising a tooth anchoring strip at the mouth end of each teeth slot.

13) The device as recited in claim 1, wherein the breathing cavity narrows from the exterior end to the teeth slots allowing for the acceptance a breathing tube having various outer diameters.

14) The device as recited in claim 1, further comprising a breathing tube.

15) The device as recited in claim 14, further comprising an air filter on the breathing tube.

16) The device as recited in claim 14, wherein the breathing tube bi-furcates into a first and second breathing tube.

17) The device as recited in claim 16, wherein the first breathing tube includes a one-way intake valve and the second breathing tube includes a one-way exhaust valve.

18) The device as recited in claim 16, wherein second breathing tube has a length extending to a lower extremity of the human body.

19) The device as recited in claim 1, wherein the mouthpiece has two breathing cavities.

20) A device for redirecting air flow from a human having a mouth, the mouth having an oral cavity, lips and teeth; comprising:
a) a mouthpiece;
b) a breathing tube emanating from the mouthpiece creating an air pathway;
c) a splitting connector integrated with the breathing tube that splits the air pathway;
d) a one-way air intake valve on one side of the split air pathway to allow air to flow into the user's mouth;
e) a one-way exhaust valve on the other side of the split air pathway to allow air to flow out of the user's mouth; and
f) an air filter integrated with the intake valve that purifies the air and excludes all contaminates from entering the user's breathing system.
